(19) United States
(12) Patent Application Publication
(10) Pub. No.: US 2001/0054421 A1
(43) Pub. Date: Dec. 27, 2001

(54) Apparatus and method for generating an aerosol of an exact dose for inhalation purposes

(75) Inventors: Stefan Jaser, Bobingen (DE); Martin Knoch, Berg (DE)

Correspondence Address: MERCHANT & GOULD PC, P.O. BOX 2903, MINNEAPOLIS, MN, 55402-0903, US

(73) Assignee: PARI Spezialisten fur effektive Inhalation, Starnberg (DE)

(21) Appl. No.: 09/911,244

(22) Filed: Jul. 23, 2001

Related U.S. Application Data
(63) Continuation of application No. 09/405,968, filed on Sep. 27, 1999, now abandoned.

(30) Foreign Application Priority Data
Oct. 2, 1998 (DE) DE 198 45 487.2

(51) Int. Cl. A61M 11/00; A61M 15/00; A61M 16/10; F24J 3/00; A62B 7/00; A61M 16/00

(52) U.S. Cl. 128/200.14; 128/203.16; 128/204.17

(57) ABSTRACT

The present invention relates to an apparatus and method for generating an aerosol of an exact dose for inhalation purposes. The inventive apparatus provides a temperature sensor 100 and/or a humidity sensor 200 in order to measure the ambient temperature of the system or the humidity of the air drawn in by the compressor 21. The sensors are preferably arranged adjacently between the outlet of a drying stage 25 and a dispersing nozzle 3. The maximum amount of a medicament that can be absorbed by a predetermined air volume can be determined (detected) by such determination of temperature and humidity. On the basis of this determination result, the supply of the appropriate amount of the medicament-containing liquid is controlled by a control means 5.

The present invention relates to an apparatus and method for generating an aerosol of an exact dose for inhalation purposes according to the features of the generic part of claim 1 and/or claim 12.

Such apparatus for generating an aerosol is for instance known from German Patent Application DE 44 38 292 A1. The purpose of the apparatus for generating an aerosol as described therein is to generate an aerosol of an exact dose to be inhaled by a patient for inhalation purposes. The known apparatus comprises a dispersing nozzle for mixing an active-substance containing liquid with a gaseous dispersion medium by forming an aerosol; a liquid supply apparatus for providing and supplying defined amounts of said active substance-containing liquid to the dispersing nozzle; a dispersion medium supply apparatus for making available and supplying the dispersion medium to the dispersing nozzle at a defined pressure; control means connected to the supply apparatuses for coordinating and controlling the supply of the dispersion medium and of the active substance-containing liquid, wherein the apparatus stands out in particular due to the use of an aerosol drying vessel for buffering and drying the aerosol generated by said dispersing nozzle, said vessel being connected to the dispersing nozzle, and a vessel provided in the liquid supply apparatus (said vessel being for example an injector or a syringe) for receiving the liquid containing the active substance, the volume of the vessel being changeable by a control means.

Furthermore, the known apparatus comprises a drying stage arranged in the dispersion medium supply apparatus. This drying stage removes moisture from the ambient air drawn in by the compressor. The compressor as well as the drying stage are arranged in the dispersion medium supply apparatus.

In order to obtain an exact dosing of liquid to be supplied to the dispersing nozzle. the liquid supply apparatus disclosed in the known apparatus for generating an aerosol stands out by a special structure. An injector to be filled with the active substance-containing liquid is provided. The injector plunger is connected to a linear feed means for moving the injector plunger into the desired position. This movement is carried out by the control means connected to the liquid supply apparatus. In this way a desired amount of said liquid can be supplied to the dispersing nozzle with a high precision.

Although in the known apparatus for generating an aerosol many efforts are taken and an exact control of the medicine-containing liquid to be supplied to the dispersing nozzle is obtained, the humidity and temperature of the ambient air taken in by the compressor or of the medium supplied to the dispersing nozzle are very inaccurate. It is true that by means of a drying stage moisture is removed from the ambient air drawn in, but exact data of humidity and temperature are not known. Therefore, the known aerosol-generating apparatus is disadvantageous in that many efforts are taken to be able to supply even the smallest amounts in a controlled manner to the dispersing nozzle, but that the ambiance conditions, especially humidity and temperature, are not known and are not taken into consideration, this disadvantage negatively influencing the precision in consistency of the drying of the aerosol and the particle size resulting therefrom.

On the basis of the afore-mentioned prior art, an improvement of the precision on consistency of the aerosol drying as well as an optimation of the medicament efficiency specific to the device is possible, which is achieved by the invention described in the following.

Consequently, the present invention is based on the object to improve the known apparatus for generating an aerosol in a way that an optimum generation and output of an aerosol with an exact dose and consistency is guaranteed.

This object is solved by an apparatus and a method comprising the features of claims 1 and/or 12. Further advantageous embodiments are defined in the dependent claims.

In this connection, one main subject of the present invention is to exactly define the ambiance conditions, especially humidity of the air drawn in and the ambient temperature of the system.

The exact ambiance conditions are necessary to achieve an optimum dosage determination, as an optimum loading with liquid of the air within a defined volume depends on these conditions. The maximum mass of medicament liquid that can be absorbed by a predetermined volume in such an apparatus for generating aerosol per atomizing cycle in order to achieve a certain final humidity of air and the particle size distributions resulting therefrom in the predetermined volume, basically depends on the ambiance conditions cited above.

To this purpose the invention provides both a sensor for temperature and a sensor for humidity positioned at an appropriate place between the outlet of the drying stage and the dispersing nozzle. Preferably the two sensors are arranged adjacently in order to enable a most exact dosing. As an alternative the temperature sensor can also be fixed in the environment of the aerosol drying vessel, especially if by means of the aerosol drying vessel a warming up of the of aerosol to body temperature (appr. 37° C.) is intended in order to further increase the water receptiveness of the air.

A further embodiment can do without such a temperature sensor. In this case the maximum amount of medicine to be absorbed is evaluated on the assumption that the ambient temperature is constant.

The invention is described in detail in the following by means of embodiments with reference to the drawings, wherein

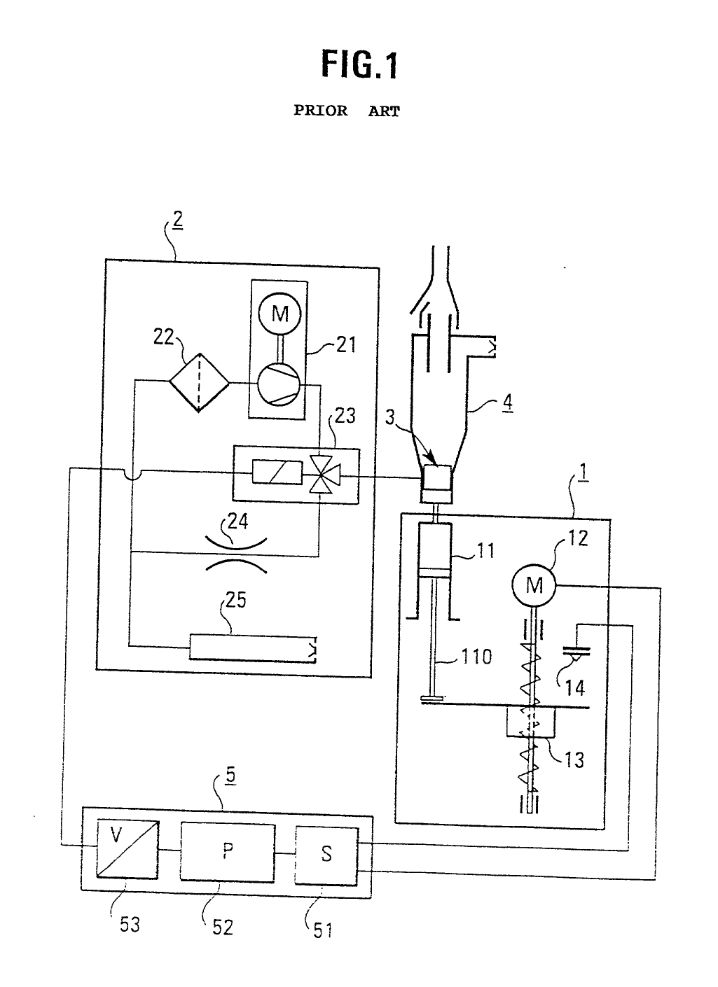

FIG. 1 schematically shows the structure of a known apparatus for generating aerosol;

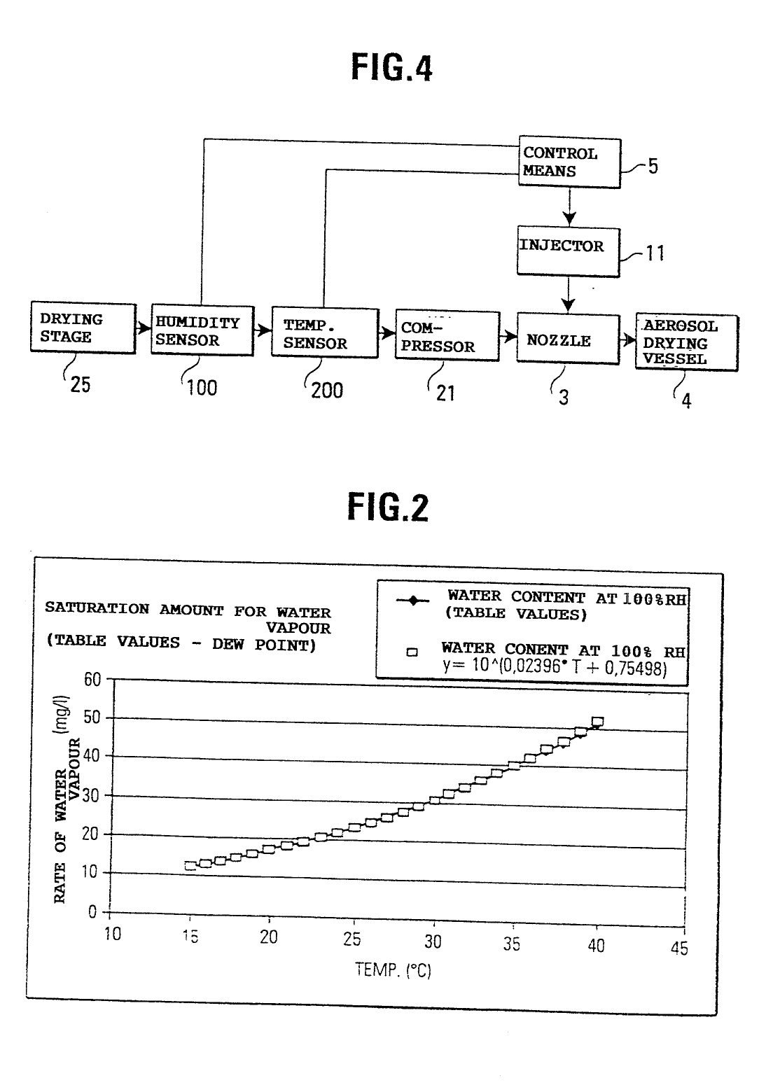

FIG. 2 shows the amount of saturation for water vapour (steam) as a function of temperature in a temperature range between 15° C. and 40° C.;

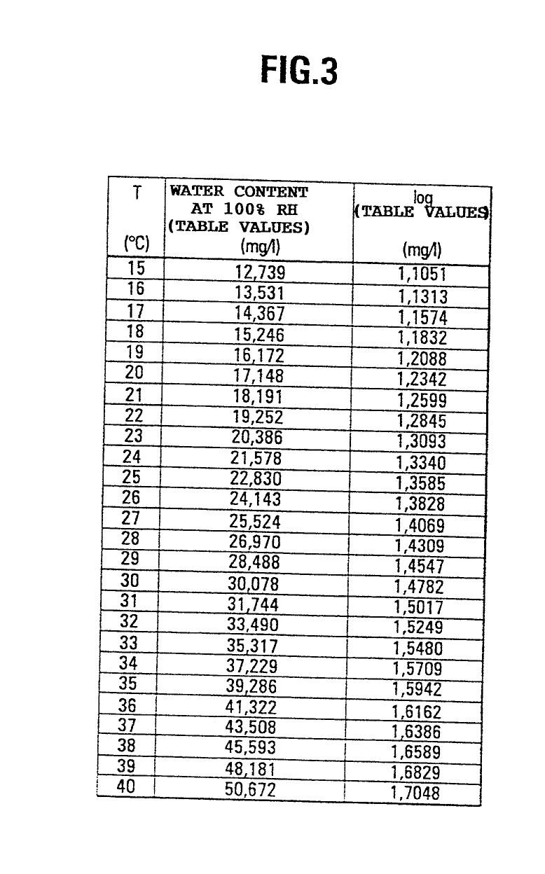

FIG. 3 shows in tabular form the values represented graphically in FIG. 2;

FIG. 4 schematically shows the principle of the present invention; and

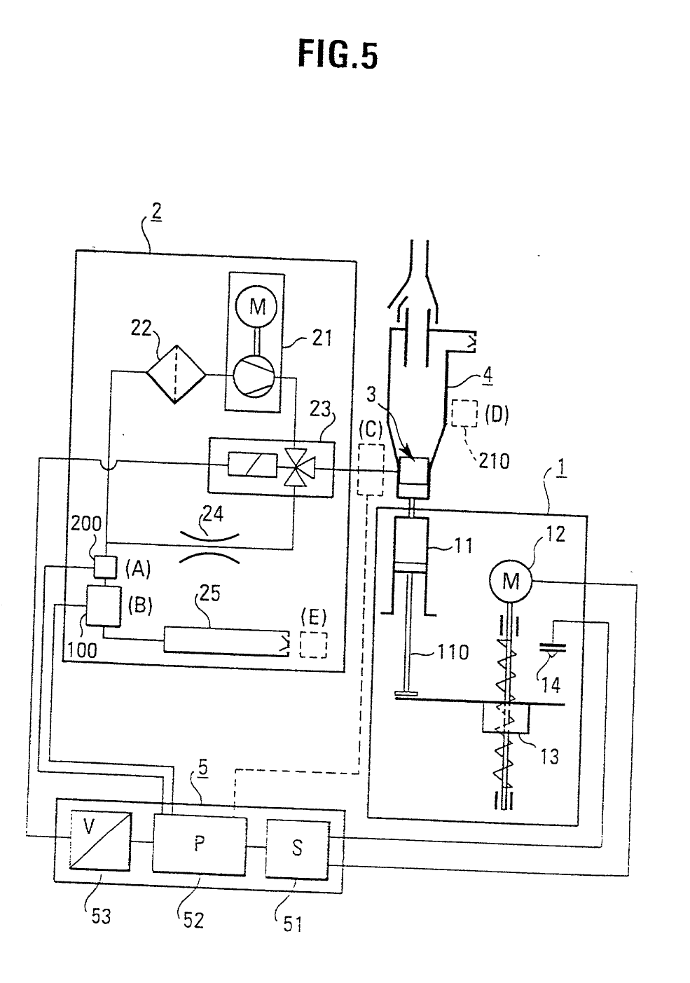

FIG. 5 schematically shows the structure of the inventive apparatus for generating aerosol, comprising a temperature sensor and/or a humidity sensor.

FIG. 1 shows the schematical structure of a known apparatus for generating aerosol. The reference sign 1 denotes a liquid supply apparatus for making available a liquid containing the active agent supplied to the dispersion nozzle 3. The dispersion medium supply apparatus 2 serves to provide the dispersion nozzle 3 with pressurized dispersion medium. In the dispersion nozzle 3 the liquid containing the active agent and the pressurized dispersion medium are mixed while forming an aerosol and sprayed into an aerosol drying vessel 4. In the aerosol drying vessel 4 the aerosol produced by the dispersion nozzle 3 can homogenize and the aerosol particles can dry up to a preferred size, the latter being regulated by the predetermined final humidity.

The dispersion medium supply apparatus 2 includes a compressor 21 with an upstream filter 22 and a controllable valve 23. This valve 23 is either connecting the outlet to the dispersing nozzle 3 or to a bypass throttle 24. Additionally, a drying stage 25 is provided which removes moisture from the ambient air drawn in by the compressor 21. For coordination and control purposes, the dispersion medium supply apparatus is connected to the control means 5.

The liquid supply apparatus includes a suitable vessel (ideally an injector or syringe 11) for receiving the medicine-containing liquid that is to be nebulised in the dispersion nozzle 3. By means of a linear feed means 13 comprising a stepping motor 12, the control means 5 controls the movement of the injector plunger 110 thus allowing a controllable feeding of the liquid stored in the injector to the dispersion nozzle 3. This control of the movement of the injector plunger allows an exact dosage of the liquid containing the medicament to be supplied to the dispersion nozzle 3. Thereby also an exact supply of even very small portions is possible.

The precision on consistency of the aerosol drying in the drying vessel 4 and the particle size distribution of the aerosol made available resulting therefrom do not only depend on the precise control of the liquid supply apparatus but also on physical variables determining the ambiance conditions of the system. The quantity of medicament m that can be absorbed by the air in the aerosol drying vessel per atomizing cycle, the volume of the drying vessel being V, this variable depending on the ambient temperature T₁ and the humidity of the air rF₁ of the taken-in air, the temperature T₂ and on the predetermined final humidity rF₂ in the aerosol drying vessel, can be evaluated with the following formulation (1):

$${m}_{( rF1 , rF2 T1 , T2 )} = ( {10}^{( aT2 + b )} \cdot \frac{{rF}_{2}}{100} - {10}^{( aT1 + b )} \cdot \frac{{rF}_{1}}{100} ) \cdot V$$

with a=0,02396 and b=0,75498

and wherein

m (rF₁, rF₂, T₁, T₂) being the mass of the liquid containing the medicament in [mg] that can be absorbed per atomizing cycle in dependency on the ambient conditions (T₁, T₂, rF₁) and the predetermined final humidity (rF₂)

T₁ being the temperature of the air drawn in in [° C.]

T₂ being the temperature of the air in the aerosol drying vessel in [° C.]

V being the volume of the aerosol drying vessel in [I]

rF₁ being the measured humidity of air of the air drawn in in [% relative humidity], and

rF₂ being the predetermined final humidity to be measured in the aerosol drying vessel after atomization in [% relative humidity].

This interrelationship applies especially for a temperature range from 15° C. up to approx. 40° C. But temperatures up to 80° C. in the aerosol drying vessel can be taken into consideration, in particular when a warming up of the aerosol in the drying vessel is wanted, in order to increase the drying effect and thus to maximize the output of a medicament liquid, the final humidity of the air being predetermined.

From the above formulation (1) results that an optimum loading of the air with liquid depends on the physical variables volume V of the aerosol drying vessel, temperature T₁ of the air drawn in, temperature T₂ of the air in the aerosol drying vessel, the output humidity rF₁ and the final humidity rF₂. The predetermined final humidity remaining in the aerosol drying vessel after atomization preferably amounts to a fixed value of 60 to 70% relative humidity in order to guarantee a complete drying of the aerosol and thus a minimum aerosol particle size, and this minimum particle size achieves a minimum deposition in the upper respiratory tracts and a maximum deposition in the lower respiratory tracts of the patient after an inhalation process.

In order to increase the quantity of medicament supplied with each inhalation operation, it is also possible to preset the final humidity of the air at rates between 70 and 100%, this not allowing the aerosol to dry completely and the droplet size being smaller than the initial one (partial drying).

In order to explain the above mentioned interdependencies and the above formulation (1), the following physical properties of the system will be explained in more detail.

To this end FIG. 2 shows the amount of saturation for water vapour as a function of temperature in the temperature range between 15° C. and 40° C. The values of the amount of saturation for water vapour at a relative humidity of 100% are known from standard table works.

With respect to the explanation of the term “saturation amount” it is stated that on vaporizing a liquid vapour is issued, the partial pressure of this vapour increasing to a certain maximum temperature-dependent value, the so called “saturation vapour pressure”. If this saturation vapour pressure is reached, it is in equipoise with the liquid pressure and the vapour is saturated. The amount (mass) of vapour contained in a certain volume is called the “saturation amount”. FIG. 2 shows the corresponding temperature-dependency. In other words, the temperature-dependency as shown in FIG. 2 describes the maximum amount /mass of water that can be comprised in pure water vapour, i.e. in a one-hundred percent vapour steam. In the kind of droplet collections necessary for inhalation purposes, the saturation vapour pressure is reached already at essentially lower relative humidity rates due to a considerable surface curvature at the contact surface between droplets and air (Kelvin effect). Therefore. the relative humidity rF₂ has to be present in the aerosol drying vessel even in case of 60 to 70% in order to achieve a complete drying of the aerosol.

The values traced in a graph in FIG. 2 are scheduled in FIG. 3. For the values as represented in FIG. 2 and 3 either a logarithmic or an exponential mean straight line can be determined.

Under reference to the values indicated in FIG. 2 and 3 and using the formulation log y=a·x+b, wherein a is the ascent of the logarithmic straight line and b is the y-axis section with x=0; and by applying the Gaussian Principle of the least squares

$$( {\sum }_{t = 1}^{n} {v}_{1}^{2} = minimum )$$

the following values for the ascent a and the y-axis section b are obtained, namely

a=0,02396 and

b=0,75498.

Thus, from the formulation

log y=0,02396x+0.75498

results the logarithmic mean straight line

y=10^{(0.02396x+0,75498)}

Analogous to the determination of the logarithmic straight line (least error squares) an exponential mean straight line for the temperature range 15° C.≦T≦40° C. can be determined with the formulation In y=cx+d. Analogous to the above observation, there result the ascent value c and the y-axis section d of

c=0,05517 and

d=1,73841.

Consequently, from

In y=0,05517x+1,73841

there is obtained the exponential mean straight line

y=e^{(0,05517x+1,73841)}

As already mentioned above, the saturation values indicated in FIGS. 2 and 3 refer to a relative humidity of 100%. In this connection it is noted that the air always contains a more or less considerable percentage of vapour. This percentage is subject to temporal and local variations and is defined as air humidity or relative humidity. Consequently, at each temperature only a maximum amount of vapour can be contained in a defined air volume. Therefore, for the determination of the vapour percentage in air the following linearity can be derived:

$${C}_{W ( \% RH )} = {C}_{W ( 100 \% )} \cdot \frac{rF}{100}$$

wherein

c_{W(%RH)}=relative mass in [mg/l] with a defined air humidity in dependency of the temperature

c_{W(100%)}=the relative mass in [mg/l] with 100% saturation degree in dependency of temperature; and

rF=the air humidity (actual degree of saturation) in [%].

Thus, from the above determined logarithmic mean straight line the following formulation results for the percentage of vapour in the air in dependency of the temperature:

$${c}_{W ( \% RH , T )} = {10}^{( 0 , 02396 T + 0 , 75498 )} \cdot \frac{rF}{100}$$

It is noted that a temperature-dependent percentage of air humidity can also be determined with the exponential mean straight line as obtained above. It is, however. not deemed necessary to describe an evaluation by means of said exponential mean straight line.

For the final calculation of the maximum amount of medicament that can be absorbed it results from the relation m_{H2O}=cw(%RH,T)V, wherein m_{H2O} is the mass in [mg] of the water absorbed in a defined volume, c_{W(%RH,T)}, as mentioned above, is the relative mass at a defined humidity of air rF and a defined temperature T, and V is the volume in consideration. The units of the values mentioned last each correspond to the ones indicated before.

Therefore, the amount of liquid required to accumulate a predetermined air volume from a given starter air humidity to a wanted final air humidity is subject to the above described dependencies.

The volume of the aerosol drying vessel can easily be determined. Using different aerosol drying vessels, the apparatus for generating an exact dose of aerosol can be adapted for instance to special patient groups (adults or children). If the aerosol drying vessel has a smaller volume, it results from formulation (1) that the maximum amount of medicament that can be absorbed is smaller than the amount of medicament that can be absorbed by means of a larger volume.

A humidity sensor 100 and a temperature sensor 200 are included in order to exactly define the humidity of the air drawn in by the compressor 21 and to exactly measure the ambient temperature of the system, as shown schematically in FIG. 4. The drying stage 25 for example is filled with a silica gel and as a rule dries the ambient air drawn in by the compressor 21 from a humidity of 45%-50% down to approximately 5% to 15%. The humidity sensor and the temperature sensor are preferably placed between the outlet of the drying stage 25 and the inlet of the compressor 21. Such an arrangement is advantageous in view of the inertia of the system and for the use of low cost unpressurized sensors. In order to guarantee a high precision of the system, humidity and temperature should be measured at the same place. With respect to the measurement of the temperature it is stated that here it is assumed that the temperature of the air in the aerosol drying vessel is the same as the temperature of the air drawn in (T₁=T₂). i.e. the moisture content in the aerosol drying vessel is laid out for the temperature taken after drying stage. Therefore, here only the temperature of the dried ambient temperature is taken. This air is then condensed (compressed) in the compressor which leads to a temperature rise. This temperature rise is compensated for on the other hand by the expansion downstream of the nozzle. The large surface of the aerosol drying vessel renders possible a further temperature balance. Such balancing of the occurring temperature differences justifies the assumption that the temperature in the aerosol drying vessel is the same as the ambient temperature.

The arrangement of the humidity sensor and the temperature sensor also follows from the structure of the present invention schematically shown in FIG. 5. The humidity sensor 100 and the temperature sensor 200 are positioned in the dispersion medium supply apparatus preferably after the drying stage 25 at the position marked (A) and (B) in FIG. 5. But the humidity sensor 100 can also be positioned at the outlet of the dispersion medium supply apparatus at the position marked (C) in FIG. 5. In this case the humidity of the dispersion medium supplied directly to the dispersion nozzle is measured. The determination of temperature is also not only restricted to measurement at position (B). The temperature sensor 200 can also be positioned directly at the aerosol drying vessel (4) (position (D) in FIG. 5) or at the drying stage 25 (position (E) in FIG. 5).

Another preferred embodiment of the invention does not provide a temperature sensor. In this case the inventive aerosol-generating apparatus can be set to a fixed, constant temperature, i.e. the apparatus is only suitable for working with a constant temperature. This can e.g. be achieved by the fact that the aerosol-generating apparatus is used in an air-conditioned room, whereby the flexibility of the inventive apparatus is restricted to a certain degree. The ambient temperature T is known for such an embodiment. Therefore, as the volume of the aerosol drying vessel can easily be determined (see before), in compliance with formulation (1) only the air humidity of the air drawn in in the dispersion medium supply apparatus remains to be determined by the humidity sensor 100 in order to determine the maximum medicine-containing liquid to be absorbed. Also in this described arrangement the humidity sensor 100 is placed between the outlet of the drying stage 25 and the dispersing nozzle 3.

Another preferred embodiment of the invention provides a heatable aerosol drying vessel for warming up the aerosol supplied. The aerosol is preferably warmed up to almost the body temperature (appr. 37° C.). Such warming up allows a further increase of the water receptiveness of the air and an optimal adjustment to the conditions in the respiratory tracts. In this case a second temperature sensor 210 must be placed near the aerosol drying vessel 4 (Position D in FIG. 5) for measuring the air temperature T₂.

In order to coordinate and control the inventive apparatus for generating an aerosol the humidity sensor 100 and the temperature sensor 200 are, as shown in FIG. 4 and 5, connected to the control means 5. The processor 52 of the control means 5 is fed with the values measured by said sensors. On the basis of the values determined by the sensors 100 and 200 and the preset final humidity, the maximum amount of medicament liquid which can be absorbed can be determined in the processor with the above mentioned formulation (1). On the basis of the determined medicament liquid amount, the liquid supply apparatus is correspondingly controlled during the dispersion process, in particular the movement of the injector plunger.

Thus, by the present invention first of all the maximum amount of medicament that can be absorbed is determined by measuring humidity and temperature. Based on this result the liquid supply apparatus is accordingly controlled by the control means 5 in order to supply the correct amount of liquid to the dispersion nozzle.

Finally it is to be pointed out that the actual (real) amount of the medicament substance can be neglected because when the air is loaded with liquid at the worst it is assumed that only water is nebulized. This may occur, as—as a rule—the medicament concentration is very small (e.g. isotonic NaCl solution: 0.9%). This assumption may improve the nebulization, as higher concentrations reduce the humidity in the spacer.

What is claimed is:

1. An apparatus for the generation of an exact dose of an aerosol, comprising:
a dispersing nozzle (3) for mixing a liquid containing a medicament with a gaseous dispersion medium;
a liquid supply apparatus (1) for supplying the liquid containing the medicament to the dispersing nozzle (3);
a dispersion medium supply apparatus (2) comprising a drying stage (25) and a compressor (21) and being connected to the dispersion nozzle (3) in order to supply the latter with the dispersion medium; and
control means (5) being connected to the supply apparatus (1, 2) characterized in that
a humidity sensor (100) is disposed between the drying stage (25) and the dispersing nozzle (3).

2. The apparatus according to claim 1, characterized in that a humidity sensor (100) and a temperature sensor (200) are disposed between the drying stage (25) and the dispersing nozzle (3).

3. The apparatus according to claim 2, characterized in that the humidity sensor (100) and the temperature sensor (200) are disposed adjacent to each other.

4. The apparatus according to claim 1, characterized in that an aerosol drying vessel (4) for drying the aerosol is provided, this aerosol drying vessel being connected to the dispersing nozzle (3).

5. The apparatus according to claim 4, characterized in that the aerosol drying vessel (4) can be heated for heating the aerosol.

6. The apparatus according to claim 4, characterized in that a second temperature sensor (210) for determining the temperature of air in the aerosol drying vessel is disposed at the aerosol drying vessel (4).

7. The apparatus according to anyone of claims 1 to 6, characterized in that the drying stage (25) contains silica gel as a drying means.

8. The apparatus according to claim 7, characterized in that the room air drawn in by the compressor (21) is dried down to 5-15% in the drying stage (25).

9. The apparatus according to anyone of claims 1 to 8, characterized in that one outlet of the humidity sensor (100) is connected to the control means (5) for controlling the liquid supply apparatus (1).

10. The apparatus according to anyone of claims 2 to 9, characterized in that one outlet of the temperature sensors (200, 210) is connected to the control means (5) for controlling the liquid supply apparatus (1).

11. The apparatus according to anyone of claims 1 to 10, characterized in that the control means (5) comprises a data processing unit (52) which receives the information from the humidity sensor (100) and from the temperature sensors (200, 210).

12. A method for the generation of an aerosol of an exact dose comprising the following steps:
mixing of a medicament-containing liquid and a gaseous dispersion substance;
supplying the dispersing nozzle (3) with the medicament-containing liquid;
supplying the dispersing nozzle (3) with the dispersing medium;
coordination and controlling of the liquid supply apparatus (1) and the dispersion medium supply apparatus (2) characterized in that
the humidity of the air drawn in by the compressor (21) is determined.

13. The method according to claim 12, characterized in that the humidity of the air drawn in by the compressor (21) and the ambient temperature of the system are determined.

14. The method according to claim 12, characterized in that the humidity and the ambient temperature of the air drawn in are determined between the drying stage (25) and the dispersing nozzle (3).

15. The method according to claim 12, characterized in that the air temperature in an aerosol drying vessel (4) is measured.

16. The method according to anyone of claims 12 to 15, characterized in that the amount of medicament to be supplied to the dispersing nozzle (3) can be determined with the formulation
$${m}_{( rF1 , rF2 , T1 , T2 )} = ( {10}^{( aT2 + b )} \cdot \frac{{rF}_{2}}{100} - {10}^{( aT1 + b )} \cdot \frac{{rF}_{1}}{100} ) \cdot V$$
wherein
m (rF₁, rF₂, T₁, T₂) stands for the mass of medicament liquid in [mg] which can be absorbed per atomizing cycle in dependency of the ambient conditions (T₁, T₂, rF₁) and the predetermined final humidity (rF₂);
T₁=the temperature of the air drawn in in [° C.];
T₂=the temperature of the air in the aerosol drying vessel in [° C.];
V=the volume of the aerosol drying vessel in [I];
rF₁=the measured air humidity of the air drawn in in [% relative humidity] and
rF₂=the predetermined final humidity in [% relative humidity] which is to be present in the aerosol drying vessel after atomization.

17. The method according to claim 16, characterized in that the variables a and b range from 0,0235 to 0,0245 or from 0,7545 to 07755.

18. The method according to claim 16, characterized in that the variable a=0,02396 and the variable b=0,75498.

19. The method according to anyone of claims 12 to 18, characterized in that the control means (5) controls the liquid supply apparatus (1) and the dispersion medium supply apparatus in accordance with the values detected by the humidity sensor (100) and the temperature sensors (200, 210).

20. The method according to anyone of claims 12 to 19, characterized in that the final humidity (rF₂) is set at 60-70% in order to completely dry the aerosol.

21. The method according to anyone of claims 12 to 19, characterized in that the final humidity (rF₂) is set at 70-100% in order to partially and consistently dry the aerosol.

22. The method according to anyone of claims 12 to 21, characterized in that the aerosol is warmed up to body temperature (approx.) in a heatable aerosol drying vessel (4).
